(19) United States
(12) Patent Application Publication
(10) Pub. No.: US 2023/0201885 A1
(43) Pub. Date: Jun. 29, 2023

(54) METHOD FOR SELECTING SCALE-DISSOLVING AGENT

(71) Applicant: FUJI ELECTRIC CO., LTD., Kawasaki-shi (JP)

(72) Inventors: Taichiro Kato, Musashino-city (JP); Kuniyuki Takahashi, Hino-city (JP); Shinya Ui, Hachioji-city (JP); Hideki Yamamoto, Osaka (JP)

(73) Assignee: FUJI ELECTRIC CO., LTD., Kawasaki-shi (JP)

(21) Appl. No.: 18/173,415

(22) Filed: Feb. 23, 2023

Related U.S. Application Data
(63) Continuation of application No. PCT/JP2022/008876, filed on Mar. 2, 2022.

(30) Foreign Application Priority Data
Mar. 25, 2021 (JP) 2021-052155

(51) Int. Cl. B08B 3/08; B08B 7/00; B08B 7/04

(52) U.S. Cl. CPC B08B 3/08; B08B 7/0071; B08B 7/0092; B08B 7/04

(57) ABSTRACT

A method provides for selecting a scale-dissolving agent suitable for components of the scale. The method for selecting the scale-dissolving agent includes a step of determining coordinates of specific physical properties of scale to be removed based on Hansen solubility parameter and a step of selecting the dissolving agent based on the distance between the coordinates of specific physical properties of scale to be removed and the coordinates of specific physical properties of the dissolving agent. Also provided is a method for removing scale using it.

### TECHNICAL FIELD

The present invention relates to a method for selecting a scale-dissolving agent and to a method for removing scale.

### BACKGROUND ART

Conventionally, scale deposition has been a problem in systems equipped with a system in which fluid is circulated, such as power plant systems, ship systems, boiler systems, and steel plant systems.

It is known that cleaning agents are used to remove scale. For example, in geothermal power plants, for the purpose of preventing operational trouble caused by generation and deposition of sulfur scale, a sulfur scale cleaning method including using hydrazine capable of dissolving elemental sulfur and a mixed aqueous solution of sodium sulfite or sodium sulfide and a wetting agent is known (for example, refer to Patent Document 1).

### REFERENCE DOCUMENT LIST

### Patent Document

Patent Document 1: JP 4-141299 A

### SUMMARY OF THE INVENTION

### Problem to be Solved by the Invention

Patent Document 1 concludes that a dissolving agent that easily dissolves elemental sulfur is effective as a dissolving agent for sulfur scale. However, in a geothermal power plant, the main component of sulfur scale covering the base material surface of a unit in which hydrogen sulfide circulates is iron sulfide. In addition, the iron sulfide surface is covered with silica scale to form a multi-layer structure. Accordingly, the scale is not dissolved with the dissolving agent disclosed in Patent Document 1, so that there is a problem in that maintenance costs increase.

Also, for example, geothermal power plants are scattered across in Japan and overseas. The components of the scale generated at each geothermal power plant are different even within Japan. Furthermore, even within the same plant, the components of the fluid which circulates in the plant differ from place to place. Therefore, it has been sometimes difficult to remove scale using only a dissolving agent capable of dissolving and removing a specific component.

Also, in various plants other than geothermal power plants in which a fluid is circulated, various types of scale are generated which are derived from the components contained in the fluid, and using only a dissolving agent capable of dissolving and removing a specific component was not sufficient.

### Means for Solving the Problem

In order to select a dissolving agent corresponding to the components of scale, the present inventors have considered selecting a dissolving agent for the scale to be removed based on a Hansen solubility parameter (HSP). A method for selecting a solvent to remove an adhesive or a peeling agent for a resin coating based on the value of the interaction distance of the HSP is known (for example, JP 2020-107754 A, JP 2015-113367 A).

However, it is not known about the selection of dissolving agents for chemicals such as scale, which deposited without artificial manipulation, being present in a heterogeneous form including unknown components, or in a multi-layer structure. As a result of further intensive research, the present inventors conceived the idea of selecting a dissolving agent corresponding to the composition and structure of the scale to be removed, and they have thereby completed the present invention.

In other words, an embodiment of the present invention relates to a method for selecting a scale-dissolving agent including: a step of determining coordinates of specific physical properties of scale to be removed based on HSP; and a step of selecting the dissolving agent based on the distance between the coordinates of the specific physical properties of the scale to be removed and the coordinates of the specific physical properties of the dissolving agent.

In the method for selecting a scale-dissolving agent, it is preferable that the specific physical properties be one or more selected from dispersion force δd, force between dipoles δp, and hydrogen bonding force δh.

In the method for selecting a scale-dissolving agent, it is preferable that the step of selecting a dissolving agent be a step of selecting a mixed dissolving agent in which two or more different chemicals are mixed, and the mixed dissolving agent have a composition determined such that coordinates of the specific physical properties of the mixed dissolving agent based on HSP approach the coordinates of the specific physical properties of the scale to be removed.

In the method for selecting the scale-dissolving agent including the step of selecting the mixed dissolving agent, it is preferable that the mixed dissolving agent be mixed with x (mol %) of chemical A and y (mol %) of chemical B, in which the step of selecting a mixed dissolving agent includes a step of determining chemical A, chemical B, x and y, such that a distance Ra₂ between coordinates of the specific physical properties of the mixed dissolving agent and coordinates of specific physical properties of the scale to be removed approaches zero:

Ra₂[4×{δdS−(δdA×x/100+δdB×y/100)}²+{δpS−(δpA×x/100+δpB×y/100)}²+{δhS−(δhA×x/100+δhB×y/100)}²]^{1/2}

in which δdS represents dispersion force, δpS represents force between dipoles, and δhS represents hydrogen bonding force of the scale to be removed, respectively;

δdA represents dispersion force, δpA represents force between dipoles, and δhA represents the hydrogen bonding force of chemical A, respectively; and

δdB represents dispersion force, δpB represents force between dipoles, and δhB represents the hydrogen bonding force of chemical B, respectively.

In the method for selecting a scale-dissolving agent, it is preferable that the step of selecting the dissolving agent be a step of selecting a single dissolving agent made of a single chemical C, such that the single chemical C has shorter distance Ra₁ between coordinates of the specific physical properties of chemical C and coordinates of specific physical properties of the scale to be removed:

Ra₁={4×(δdS'δdC)²+(δpS−δpC)²+(δhS−δhC)²}^{1/2}

in which δdS represents dispersion force, δpS represents force between dipoles, and δhS represents hydrogen bonding force of the scale to be removed, respectively; and

δhC represents dispersion force, δpC represents force between dipoles, and δhC represents the hydrogen bonding force of chemical C, respectively.

In the method for selecting a scale-dissolving agent, it is preferable that the step of determining coordinates of specific physical properties of the scale to be removed based on HSP include a step of collecting the scale for determining coordinates of the specific physical properties.

In the method for selecting a scale-dissolving agent, it is preferable that the step of determining coordinates of specific physical properties of the scale to be removed based on HSP include a step of determining coordinates of the specific physical properties based on elemental analysis of the fluid containing based chemicals of scaling.

In the method for selecting a scale-dissolving agent, it is preferable that the elemental analysis be a method using Inductively Coupled Plasma (ICP).

In the method for selecting a scale-dissolving agent, it is preferable that the scale to be removed contain iron sulfide and the chemical C be acetic acid, and/or that the scale to be removed contain silica and the chemical C be hydrofluoric acid.

Another embodiment of the present invention relates to a method for removing scale including a step of selecting a scale-dissolving agent based on any of the methods for selecting a scale-dissolving agent described above; and

a step of applying the scale-dissolving agent obtained in the selecting step to the scale to be removed.

It is preferable that the method for removing scale further include a step of removing the scale by a physical method in addition to the step of applying the scale-dissolving agent to the scale to be removed.

In the method for removing scale, it is preferable that the physical method include:

a step of applying a temperature change to the scale to be removed to generate a shearing force, and/or

a step of applying a mechanical force to the scale to be removed.

In the method for removing scale, the scale to be removed including two or more layers composed of different components, it is preferable that

the step of selecting a scale-dissolving agent include a step of selecting scale-dissolving agents individually for the respective two or more layers; and

the step of applying the scale-dissolving agent to the scale to be removed includes a step of sequentially applying the individually selected scale-dissolving agents to the respective two or more layers, or a step of preparing a mixture of the individually selected scale-dissolving agents for applying to the two or more layers.

Another embodiment of the present invention relates to a method for producing a mixed dissolving agent including a step of selecting a mixed dissolving agent based on the method for selecting a mixed dissolving agent described above, and a step of preparing the mixed dissolving agent.

### Effects of the Invention

According to the method for selecting a scale-dissolving agent of the present invention, a dissolving agent corresponding to the components of the scale to be removed can be selected. Even for scale generated in various types of plants and scale deposited at different places in a same plant, a dissolving agent may be selected according to the properties of the scale. In addition, it becomes possible to select and produce a dissolving agent containing multiple components. Furthermore, an effective method for removing scale can be provided using these dissolving agents.

### BRIEF DESCRIPTION OF THE DRAWINGS

FIG. 1 is a diagram illustrating a method for selecting a single dissolving agent in a first aspect of a first embodiment of the present invention, showing coordinates of specific physical properties based on HSP.

FIG. 2 is a schematic cross-sectional view showing a laminate form of scale.

FIG. 3 is a diagram illustrating a method for selecting a mixed dissolving agent in a second aspect of a first embodiment of the present invention, showing coordinates of specific physical properties based on HSP.

### MODE FOR CARRYING OUT THE INVENTION

Embodiments of the present invention will be described below with reference to the drawings. However, the present invention is not limited to the embodiments described below.

### First Embodiment

Selection of Dissolving Agent

A first embodiment of the present invention relates to a method for selecting a dissolving agent. The selection method includes the following steps:

(1) a step of determining coordinates of specific physical properties of scale to be removed based on HSP; and

(2) a step of selecting the dissolving agent based on the distance between the coordinates of specific physical properties of the scale to be removed and the coordinates of specific physical properties of the dissolving agent.

In the present invention, the dissolving agent is a chemical capable of reducing the amount of deposited scale by dissolving the scale to be removed, and it may be made of a single chemical or a mixture of two or more chemicals. A dissolving agent made of a single chemical is also referred to herein as a single dissolving agent. A dissolving agent made of a mixture of two or more chemicals is also referred to as a mixed dissolving agent.

The scale to be removed may be any scale that may contain inorganic compounds and organic compounds. More specifically, the scale may be one that is generated to deposit, being derived from chemicals dissolved in fluid chemicals such as water circulating in power plant systems such as geothermal power plant systems, thermal power plant systems, nuclear power plant systems, hydraulic power plant systems, or biomass power plant systems; ship systems such as ship exhaust gas cleaning systems (EGCS) and seawater desalination systems; boiler systems such as factory heating sources and building heating and hot water supply systems; and steel plant systems such as cooling water systems and cleaning water systems, and the types thereof are not particularly limited. For example, in a geothermal power plant, the scale may be a multi-component scale generated in layers on base materials such as pipes, heat exchangers, turbines, and drains that constitute the plant.

The specific physical properties based on HSP may be one or more selected from dispersion force δd, force between dipoles δp, and hydrogen bonding force δh. Therefore, the selection method may be based on a one-dimensional coordinate consisting of any one specific physical property, may be based on two-dimensional coordinates consisting of two specific physical properties, or may be based on three-dimensional coordinates consisting of three physical properties.

The method for selecting a dissolving agent according to the present embodiment will be described separately for the selection of a single dissolving agent and for the selection of a mixed dissolving agent in the following.

### First Aspect: Selection of Single Dissolving Agent

A first aspect of the selection method according to the present embodiment relates to a method for selecting a single dissolving agent. The selection method for a single dissolving agent includes the following steps:

(A) a step of determining coordinates of specific physical properties of a scale to be removed based on HSP; and

(B) a step of selecting a single dissolving agent made of a single chemical C based on the distance from the coordinates of the specific physical properties of the scale to be removed, in which the single chemical C has shorter distance Ra₁ between coordinates of the specific physical properties of the chemical C and coordinates of specific physical properties of the scale to be removed:

Ra₁={4×(δdS−δdC)²+(δpS−δpC)²+(δhS−δhC)²}^{1/2} (1)

in which δdS represents dispersion force, δpS represents force between dipoles, and δhS represents hydrogen bonding force of the scale to be removed, respectively; and

δdC represents dispersion force, δpC represents force between dipoles, and δhC represents the hydrogen bonding force of chemical C, respectively.

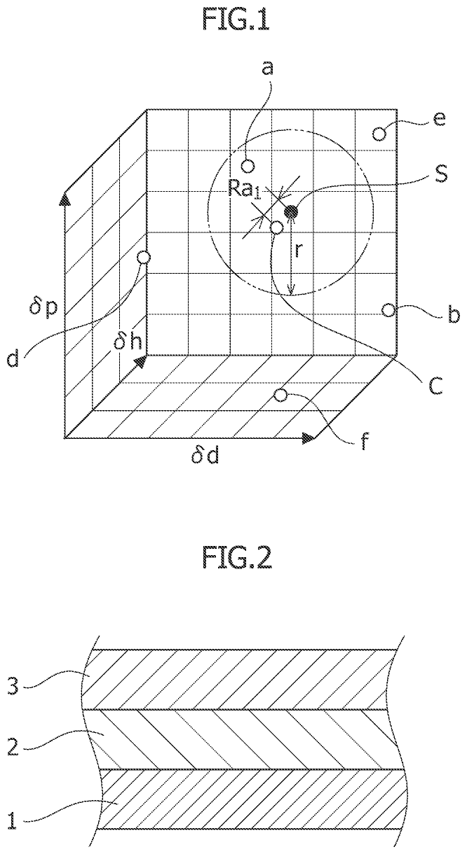

In step (A), the coordinates of the specific physical properties of the scale to be removed are determined based on HSP. FIG. 1 is a diagram conceptually illustrating a three-dimensional coordinate space used in the method for selecting a single dissolving agent. In the coordinate space, three axes of dispersion force δd, force between dipoles δp, and hydrogen bonding force δh are set, and the coordinates S of the scale plotted in the space is determined in step (A).

The coordinates δdS, δpS and δhS of the specific physical properties of the scale to be removed may be generally determined by experimentation. For example, a step of collecting the scale to be removed at a desired location of a target plant and analyzing the layer structure and components of the scale, and a step of experimentally determining the coordinates of specific physical properties from the component are included.

FIG. 2 is a diagram schematically showing the structure of scale deposited atpipes and equipment of a geothermal power plant. In a geothermal power plant, water with silica and sulfur dissolved circulates, and a layered scale may be deposited. With reference to FIG. 2, a first layer 2 made of sulfur scale containing iron sulfide as main component is formed on a base material 1 made of steel or the like, and a second layer 3 made of silica scale containing silica as the main component is formed on the first layer 2. It should be noted that FIG. 2 is a schematic diagram, and an actual scale may have uneven layer thicknesses or may have discontinuities in the middle of the layer. Moreover, the generated scale may have different components depending on the material of the base material. Furthermore, even within the same plant, susceptibility to scale generation and rate of growth may differ depending on the location. Therefore, it is preferable to analyze such a layered structure and the components of each layer corresponding to the place in which a scale to be removed is deposited. As shown in FIG. 2, in the case in which scale is composed of a plurality of layers with different main components, dissolving agents suitable for the respective layers may be selected.

It is also possible to estimate the coordinates δdS, δpS and δhS of the specific physical properties of the scale to be removed from the composition ratio of the components of the fluid circulated through the plant. The estimation is useful when it is difficult to stop the operation of the plant system, when scale cannot be collected by peeling off due to strong deposition, or when it is necessary to easily determine the composition of the scale. Since the composition of fluid circulating through the plant varies depending on the location in the plant, the fluid may be collected at any desired location. For example, in a geothermal power plant, liquid circulated through a production well, an injection well, a heat exchanger, etc., may be collected.

The collected fluid may be analyzed by any analytical method. The analytical method may be based on trace elemental analysis. For example, elemental analysis may be performed by a method using Inductively Coupled Plasma, and more specifically, ICP-MS analysis may be employed, although this is not limited to a specific method.

The step of experimentally determining coordinates of the specific physical properties from the actually collected scale components or from the results of elemental analysis of the fluid may be performed, for example, by a permeation rate method in which a solvent permeates scale particles for evaluation of affinity.

Subsequently, in step (B), a dissolving agent is selected based on the distance from the coordinates of the specific physical properties of the scale to be removed. More specifically, chemical C having coordinates of the specific physical properties at a close distance Ra₁ to the coordinates of the specific physical properties of the scale to be removed is selected.

HSPs of chemicals may be determined from a database. Also, in the case in which the structure of a chemical is known, the HSP of the chemical may be determined using a HSP software HSPiP (Hansen Solubility Parameter in Practice).

In the coordinate space shown in FIG. 1, the coordinates of six candidate chemicals a, b, C, d, e, and f are plotted. Among them, for example, chemical C with the closest distance Ra₁ may be selected as the dissolving agent of scale S. Among these chemicals, chemicals having coordinates of specific physical properties close to the coordinates (δdS, δpS, δhS) of the specific physical properties of scale S to be removed may be said to be good dissolving agents for the scale S, and chemicals having distant coordinates of specific physical properties may be said to be poor dissolving agents for the scale S. Chemical C with a shorter distance Ra₁ is most preferred as the dissolving agent.

In FIG. 1, assuming a sphere with a radius r centered on the coordinates (δdS, δpS, δhS) of the specific physical properties of scale S to be removed in the coordinate space, it can be said that any chemical having coordinates (δdC, δpC, δhC) in a sphere within the predetermined radius r is able to dissolve the removal target scale S with high efficiency. The radius r is preferably 10 (MPa^{1/2}) or less, is preferably as short as possible, and is most preferably close to zero. In other words, it can be said that a chemical having coordinates of specific physical properties with a distance Ra₁ to the coordinates of specific physical properties of scale S to be removed of 10 (MPa^{1/2}) or less is a good dissolving agent. However, in the case in which no chemical having coordinates of specific physical properties with a distance Ra₁ of 10 (MPa^{1/2}) or less is present, a chemical having the shortest distance Ra₁ may be selected as a dissolving agent, even though the chemical has an Ra₁ of more than 10 (MPa^{1/2}).

Alternatively, a plurality of indexes such as price, handleability, etc., may be provided to comprehensively select the chemical for selection of the dissolving agent instead of using distance Ra₁ only as an index.

In FIG. 1, a three-dimensional coordinate space is used as an example for explanation. Alternatively, in two-dimensional coordinates, two of δd, δp and δh may be selected for each of the scale and dissolving agent to determine Ra₁ in a method similar to that for selection of the chemical. In the formula (1), the two-dimensional coordinates may be presumed to be the case in which any one of δdS−δdC, δpS−δpC, and δhS−δhC is zero or is close to zero, that is, the case in which any of the specific physical properties is the same between the scale and the dissolving agent. Similarly, in a one-dimensional coordinate, one of δd, δp, and δh may be selected for each of the scale and dissolving agent to determine Ra₁ in a manner similar for selection of the chemical. In the formula (1), the one-dimensional coordinates may be presumed to be the case in which any two of δdS−δdC, δpS−δpC, and δhS−δhC are zero or are close to zero, that is, the case in which any two of the specific physical properties are the same between the scale and the dissolving agent.

As an example, in the case in which scale S to be removed is silica scale mainly composed of silica (SiO₂), among a plurality of candidate chemicals, hydrofluoric acid may be extracted as a chemical having a short coordinate distance Ra₁ for use as a single dissolving agent. When the coordinates of the specific physical properties of silica scale are plotted as shown in FIG. 1, the coordinates of the specific physical properties of hydrofluoric acid are present at a position in which Ra₁ is less than 10 (MPa^{1/2}) (not shown in drawing).

As another example, in a case in which scale S to be removed is sulfur scale mainly composed of iron sulfide (FeS), among a plurality of candidate chemicals, acetic acid is extracted for use as a single dissolving agent as the chemical with the shortest coordinate distance Ra₁. When the coordinates of the specific physical properties of sulfur scale are plotted as shown in FIG. 1, the coordinates of the specific physical properties of acetic acid are present at positions in which Ra₁ is less than 10 (MPa^{1/2}) (not shown in drawing).

### Second Aspect: Selection of Mixed Dissolving Agent

A second aspect of the selection method according to the present embodiment relates to a method for selecting a mixed dissolving agent. The selection method for a mixed dissolving agent includes the following steps:

(a) a step of determining coordinates of specific physical properties of scale to be removed based on HSP; and

(b) a step of selecting a mixed dissolving agent of mixture of two or more different chemicals based on the distance from the coordinates of the specific physical properties of the scale to be removed including:

determining the composition of the mixed dissolving agent such that the coordinates of the specific physical properties of the mixed dissolving agent based on the HSP approach the coordinates of the specific physical properties of the scale to be removed.

Also, in the second aspect, step (a) may be performed in the same manner as in step (A) in the first aspect, so that explanation is omitted here.

In step (b), a plurality of chemicals to constitute the mixed dissolving agent and the mixing ratio thereof are determined such that a distance Ra₂ between the coordinates of the specific physical properties of the scale to be removed and the coordinates of the specific physical properties of the mixed dissolving agent based on HSP is closer.

For example, in the case in which a mixed dissolving agent is composed of two types of chemicals A and B, distance Ra₂ in the three-dimensional coordinates is defined and explained as follows.

When x (mol %) of chemical A and y (mol %) of chemical B are mixed (in which x+y=100),

a distance Ra₂ between coordinates of the specific physical properties of the mixed dissolving agent and coordinates of specific physical properties of the scale to be removed may be expressed as follows:

Ra₂=[4×{δdS−(δA×x/100+δdB×y/100)}²+{δpS−(δpA×x/100+δpB×y/100)}²+{δhS−(δhA×x/100+δhB×y/100)}²]^{1/2} (2)

in which δdS represents dispersion force, δpS represents force between dipoles, and δhS represents hydrogen bonding force of the scale to be removed, respectively;

δdA represents dispersion force, δpA represents force between dipoles, and δhA represents the hydrogen bonding force of chemical A, respectively; and

δdB represents dispersion force, δpB represents force between dipoles, and δhB represents the hydrogen bonding force of chemical B, respectively.

Chemical A and chemical B are selected such that a line segment connecting the coordinates of the respective specific physical properties has an intersection point or a contact point with a sphere having radius r centered at the coordinates of the specific physical properties of the scale to be removed. The value of radius r may be about 10 (MPa^{1/2}), or it may be set to a value exceeding 10 (MPa^{1/2}). The coordinates of the specific physical properties of chemical A and chemical B may be determined in the same manner as in the selection of candidate chemicals in step (B) in the first aspect. Subsequently, the values of x and y are determined such that Ra₂ approaches zero for the selection of the components of the mixed dissolving agent and the composition thereof.

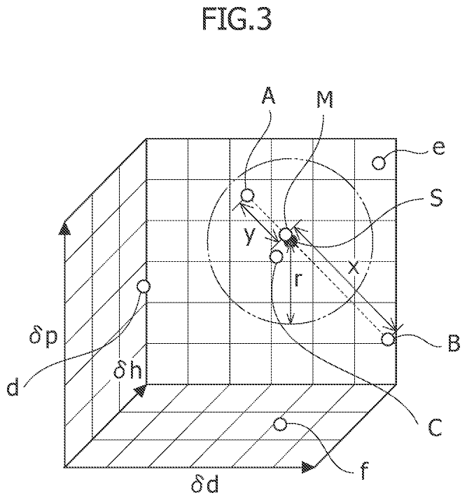

FIG. 3 is a diagram conceptually showing a three-dimensional coordinate space used in the method of selecting a mixed dissolving agent. In step (a), the coordinates S of the specific physical properties of the scale plotted in this space is determined. Six chemicals A, B, C, d, e, and f are plotted in the coordinate space shown in FIG. 3. Among these, chemical C is a single dissolving agent selected in the first aspect for the same scale to be removed. A line segment connecting the coordinates of the specific physical properties of the chemicals A and B has an intersection with a sphere having a radius of r=10 (MPa^{1/2}) centered on the coordinates S of the specific physical properties of the scale. Therefore, candidate chemicals A and B may be components of the mixed dissolving agent for scale S. Then, by applying the coordinates of the specific physical properties of the candidate chemicals A and B to formula (2), it is possible to calculate values of x and y for Ra₂ to approach zero. Coordinates M of the specific physical properties of the mixed dissolving agent may thus be determined. That is, it is possible to select a mixed dissolving agent containing chemical A and chemical B at a molar ratio of x:y.

In the second aspect also, it can be said that a mixed dissolving agent having a distance Ra₂ between coordinates S of the specific physical properties of scale S to be removed and coordinates M of the specific physical properties of the mixed dissolving agent of 10 (MPa^{1/2}) or less is a good dissolving agent. However, in the case in which chemicals A and B, and ratios x and y which allow the composition of a mixed dissolving agent to have coordinates of specific physical properties with distance Ra₂ of 10 (MPa^{1/2}) or less are not present, a chemical having the shortest distance Ra₂ may be selected as a dissolving agent, even though the chemical has an Ra₂ of more than 10 (MPa^{1/2}).

Here, it can be said that a mixed dissolving agent containing chemical A and chemical B at a molar ratio of x:y is more advantageous than a single dissolving agent made of chemical C in the following cases:

Price of mixed dissolving agent<Price of single dissolving agent, or

Ra₂ of mixed dissolving agent<Ra₁ of single dissolving agent.

Accordingly, the selection method of the present invention further includes a method comprising a step of comparing the selection method in the first aspect and the selection method in the second aspect in combination.

Although FIG. 3 and formula (2) illustrate the selection of a mixed dissolving agent of two components, a dissolving agent containing three or more components may also be selected in the same manner by extending the formula of Ra₂. In that case, for example, a plurality of candidate chemicals selected from a database are plotted around the coordinates of the specific physical properties of the scale to be removed, and the three or more chemicals to be mixed may be appropriately selected based on the plotted results by one skilled in the art. Subsequently, the molar ratio of the selected chemicals is set to x:y:z in the case of three chemicals, and a formula for the distance Ra₃ in the case of three components is created in the same manner as in formula (2). Values of x, y, and z may then be determined such that Ra₃ is zero or approaches zero. In the case of four or more chemicals, the same procedure also may be performed. In other words, the mixed dissolving agent may be selected by determining the chemicals constituting the mixed dissolving agent and the mixing ratio.

As described above, the selection method in the second aspect of the present embodiment can select a good dissolving agent made of a plurality of chemicals that would not be good dissolving agents alone. Accordingly, choices of dissolving agents drastically increase, so that maintenance time and costs of a plant having a problem of scale deposition may be reduced. Furthermore, with a combination use of the selection method in the first aspect and the selection method in the second aspect, a dissolving agent more suitable for use may be selected.

### Second Embodiment

Production Method of Mixed Dissolving Agent

The second embodiment of the present invention relates to a production method for a mixed dissolving agent. The production method for a mixed dissolving agent includes the following steps:

(i) a step of selecting a mixed dissolving agent based on the selection method described in the second aspect of the first embodiment, and

(ii) a step of mixing two or more different chemicals selected in the step described above to prepare the mixed dissolving agent.

Step (i) of the present embodiment may be performed in the same manner as in the second aspect of the first embodiment, and description thereof is omitted here. Step (ii) may be performed by mixing two or more chemicals selected in step (i) at the ratio determined in step (i).

According to the present embodiment, a mixed dissolving agent may be produced corresponding to the components of the scale, so that maintenance time and maintenance costs of a plant may be reduced. In particular, even in the absence of a suitable single dissolving agent, a mixed dissolving agent usable for removal of scale may be produced by the production method of the present embodiment.

### Third Embodiment

Scale Removal Method

The third embodiment of the present invention relates to a method for removal of scale. The method for removal of scale includes the following steps:

(I) a step of selecting a scale-dissolving agent based on the selection method described in the first embodiment; and

(II) a step of applying the scale-dissolving agent determined in the step described above to the scale to be removed.

Alternatively, the following step may be further included as an optional step:

(III) a step of removing the scale by a physical method in addition to the step of applying the scale-dissolving agent to the scale to be removed.

Step (I) of the present embodiment may be performed by the method described in the first embodiment, so description thereof is omitted here.

In step (II), the dissolving agent selected in step (I) is prepared and applied to the scale to be removed. Examples of the application method include spraying the dissolving agent on the scale to be removed, washing equipment to which the scale to be removed has deposited with the dissolving agent, contacting the dissolving agent with the scale to be removed for a predetermined period of time, blowing the dissolving agent on the scale to be removed, injecting the dissolving agent into the scale to be removed, and applying the dissolving agent to the scale to be removed by any of the methods described above, and then applying ultrasonic waves for internal permeation, although they are not limited thereto.

Step (III) is a step of applying a physical force to the scale, and it may be used in combination with the dissolving agent. In chronological order, application of the dissolving agent and the physical method may be performed simultaneously, or either thereof may be performed first. Preferably, the physical method may be performed after application of the dissolving agent to the scale to be removed.

Specific examples of the physical method include applying temperature change to the scale to be removed to generate shearing force. In the process, the temperature change is applied to the scale and a member on which scaling is found, and the shearing force generated due to the difference in linear expansion coefficient along with the temperature change causes cracks and flaws in the scale, allowing easy peeling off. For example, heating and cooling the scale and surrounding members may be performed. The heating and cooling may be repeated. These operations are advantageous in that the generated thermal stress may be maximized preferably by applying an increased temperature difference to the scale and its surrounding members. The heating may be performed using one or more means selected from heaters, induction heating (IH), microwaves, burners, boiler steam, and hot air. The cooling may be performed using one or more means selected from chillers, river water, mist spraying, and cold air.

Other examples of a specific physical method include applying mechanical force to the scale to be removed. In the process, using a tool or the like, mechanical operation such as one or more of grinding, machining, peeling, drilling, striking, vibrating (vibrators or ultrasonic waves), cutting, ripping off, and crushing may be applied to the scale to be removed. A plurality of different physical methods may be used in combination, and the physical methods are not limited to the exemplified methods.

In the case in which the scale to be removed has a multilayered structure including two or more layers made of different components, the scale may be optionally removed based on data on layer structure and components in the thickness direction of the scale to be removed determined in step (A) or step (a) in the selection method of a dissolving agent. Specifically, scale-dissolving agents individually selected for the respective two or more layers may be sequentially applied to the two or more layers, or the individually selected scale-dissolving agents may be mixed for preparation to be applied to the two or more layers. The mixing ratio in the case of a mixed preparation may be determined based on the ratios of thickness, mass, volume, etc., of two or more layers.

By the method for removal of scale according to the present embodiment, scale may be effectively and economically removed by use of an appropriate dissolving agent selected for the scale to be removed.

### EXAMPLE

The present invention will be described in detail below with reference to an Example. However, the following Example does not limit the invention.

A single dissolving agent was selected using three-dimensional coordinates according to the procedure described in the first aspect of the first embodiment of the present invention. The scale to be removed was assumed to be sulfur scale (FeS). The coordinates of the specific physical properties of the scale to be removed were experimentally determined by a permeation rate method. As a result of calculation using formula (1), a value of Ra₁ of 1.6 was obtained for sulfur scale, and acetic acid was selected as the dissolving agent. The coordinates of the specific physical properties of the selected acetic acid were δd of 14.5, δp of 8.0, and δh of 13.5.

### INDUSTRIAL APPLICABILITY

The method for selecting a scale-dissolving agent, the method for removing scale, and the method for producing a scale-dissolving agent according to the present invention may be used in the removal scale in various plant systems.

What is claimed is:

1. A method for selecting a scale-dissolving agent, comprising:
a step of determining coordinates of specific physical properties of scale to be removed based on Hansen solubility parameter; and
a step of selecting the dissolving agent based on the distance between the coordinates of specific physical properties of the scale to be removed and the coordinates of specific physical properties of the dissolving agent.

2. The selection method according to claim 1, wherein the specific physical properties are one or more selected from dispersion force δd, force between dipoles δp, and hydrogen bonding force δh.

3. The selection method according to claim 1,
wherein the step of selecting a dissolving agent is a step of selecting a mixed dissolving agent in which two or more different chemicals are mixed, and
the mixed dissolving agent has a composition determined such that coordinates of the specific physical properties of the mixed dissolving agent based on Hansen solubility parameter approach the coordinates of the specific physical properties of the scale to be removed.

4. The selection method according to claim 3,
wherein the mixed dissolving agent is mixed with x (mol %) of chemical A and y (mol %) of chemical B,
wherein the step of selecting a mixed dissolving agent comprises a step of determining chemical A, chemical B, x and y, such that a distance Ra₂ between coordinates of the specific physical properties of the mixed dissolving agent and coordinates of specific physical properties of the scale to be removed approaches zero:
Ra₂=[4×{δdS−(δdA×x/100+δdB×y/100)}²+{δpS−(δpA×x/100+δpB×y/100)}²+{δhS−(δhA×x/100+δhB×y/100)}²]^{1/2}
wherein δdS represents dispersion force, δpS represents force between dipoles, and δhS represents hydrogen bonding force in the scale to be removed;
δdA represents dispersion force, δpA represents force between dipoles, and δhA represents the hydrogen bonding force of chemical A; and
δdB represents dispersion force, δpB represents force between dipoles, and δhB represents the hydrogen bonding force of chemical B.

5. The selection method according to claim 1,
wherein the step of selecting the dissolving agent is a step of selecting a single dissolving agent made of single chemical C, such that the single chemical C has shorter distance Ra₁ between coordinates of the specific physical properties of chemical C and coordinates of specific physical properties of the scale to be removed:
Ra₁={4×(δdS−δdC)²+(δpS−δpC)²+(δhS−δhC)²}^{1/2}
wherein δdS represents dispersion force, δpS represents force between dipoles, and δhS represents hydrogen bonding force of the scale to be removed, and
wherein δdC represents dispersion force, δpC represents force between dipoles, and δhC represents the hydrogen bonding force of chemical C.

6. The selection method according to claim 1, wherein the step of determining coordinates of specific physical properties of scale to be removed based on Hansen solubility parameter comprises a step of collecting scale for determining coordinates of the specific physical properties.

7. The selection method according to claim 1, wherein the step of determining coordinates of specific physical properties of scale to be removed based on Hansen solubility parameter comprises a step of determining coordinates of the specific physical properties based on the elemental analysis of the fluid containing based chemicals of scaling.

8. The selection method according to claim 7, wherein the elemental analysis is a method using inductively coupled plasma.

9. The selection method according to claim 5,
wherein the scale to be removed contains iron sulfide and the chemical C is acetic acid, and/or
wherein the scale to be removed contains silica and the chemical C is hydrofluoric acid.

10. A method for removing scale, comprising:
a step of selecting a scale-dissolving agent based on methods for selecting a scale-dissolving agent according to claim 1; and
a step of applying the scale-dissolving agent obtained in the selecting step to scale to be removed.

11. The method for removing scale according to claim 10, further comprising a step of removing the scale by a physical method in addition to the step of applying the scale-dissolving agent to scale to be removed.

12. The method for removing scale according to claim 11, wherein the physical method comprises:
a step of applying a temperature change to scale to be removed to generate a shearing force and/or,
a step of applying a mechanical force to scale to be removed.

13. The method for removing scale according to claim 1,
wherein scale to be removed comprises two or more layers composed of different components,
the step of selecting a scale-dissolving agent comprises a step of selecting scale-dissolving agents individually for the each of two or more layers; and
the step of applying the scale-dissolving agent to scale to be removed comprises a step of sequentially applying the individually selected scale-dissolving agents to the each of two or more layers, or a step of preparing a mixture of the individually selected scale-dissolving agents for applying to the two or more layers.

14. A method for producing a mixed dissolving agent, comprising:
a step of selecting a mixed dissolving agent based on the method for selecting a scale-dissolving agent according to claim 3, and
a step of preparing the mixed dissolving agent.
